(19) United States
(12) Patent Application Publication
(10) Pub. No.: US 2025/0058051 A1
(43) Pub. Date: Feb. 20, 2025

(54) ERGONOMIC HANDLES FOR MEDICAL DEVICES

(71) Applicant: Boston Scientific Medical Device Limited, Galway (IE)

(72) Inventor: Yong Geun SEO, Seongnam-si (KR)

(73) Assignee: Boston Scientific Medical Device Limited, Galway (IE)

(21) Appl. No.: 18/804,602

(22) Filed: Aug. 14, 2024

Related U.S. Application Data
(60) Provisional application No. 63/520,147, filed on Aug. 17, 2023.

(51) Int. Cl. A61M 5/31

(52) U.S. Cl. CPC A61M 5/3137; A61M 2205/586

(57) ABSTRACT

A medical device handle may comprise a first body defining a lumen; and a second body having a portion that is movably received within the lumen. The second body may include a grip. The grip may include a proximal portion, a distal portion, and a middle portion between the proximal portion and the distal portion. The middle portion may have a greater cross-sectional width than the proximal portion and the distal portion. The second body may be configured to move proximally and distally along a longitudinal axis of the second body in order to actuate a needle of a medical device.

### CROSS-REFERENCE TO RELATED APPLICATIONS

This application claims the benefit of priority of U.S. Provisional Patent Application No. 63/520,147, filed Aug. 17, 2023, the entirety of which is incorporated herein by reference.

### TECHNICAL FIELD

Various aspects of this disclosure relate generally to ergonomic handles for medical devices. In particular, the disclosure pertains to handles for injection needles having ergonomic features.

### BACKGROUND

Injection needles may include a handle for gripping by an operator and a shaft for insertion into a body of a subject (e.g., via an insertion device such as a scope). A lumen of the shaft may receive a needle that may be selectively advanced or retracted from a distal tip of the shaft. The needle may define a lumen through which a fluid, such as contrasting agent, saline, or another agent (e.g., a lifting agent) may be injected. The needle may have a distal opening that may be used to deliver the fluid to a treatment site within a body of the subject.

The handle may include components for controlling the needle (e.g., advancing and retracting the needle), as well as components for coupling to a source of fluid. A need exists for ergonomic handles for medical devices, such as injection needles.

### SUMMARY

Each of the aspects disclosed herein may include one or more of the features described in connection with any of the other disclosed aspects. Aspects of the disclosure may relate to handles for medical devices, such as injection needles. The handles may have ergonomic features to facilitate gripping by an operator and/or repeated extension/retraction of the needle.

In an example, a medical device handle may comprise a first body defining a lumen; and a second body having a portion that is movably received within the lumen. The second body may include a grip. The grip may include a proximal portion, a distal portion, and a middle portion between the proximal portion and the distal portion. The middle portion may have a greater cross-sectional width than the proximal portion and the distal portion. The second body may be configured to move proximally and distally along a longitudinal axis of the second body in order to actuate a needle of a medical device.

Any of the devices disclosed herein may include any of the following features, alone or in any combination or subcombination. The grip may include a plurality of ridges. The ridges may extend at a non-zero angle to the longitudinal axis. The ridges may be non-perpendicular to the longitudinal axis. The longitudinal axis may be a central longitudinal axis. A first ridge of the plurality of ridges may have a first portion that extends a first radial distance from the central longitudinal axis. The first ridge may have a second portion that extends a second radial distance from the central longitudinal axis. The second radial distance may differ from the first radial distance. The first body may include a flange at a proximal end thereof. The flange may be a first flange. The first body may include a second flange that is proximal of the first flange. The first flange may have a curved surface that faces distally and the second flange may have a curved surface that faces proximally. A proximal end of the second body may include an adapter for coupling to a fluid source. The adapter may include an opening defined by a first wall, a second wall surrounding the first wall, and at least one fin extending between the first wall and the second wall. The at least one fin may include four fins that are evenly spaced from one another. The at least one fin may extend radially outward from the first wall. The adapter may include a concave proximal surface. The first body may include a barrel having a plurality of ridges thereon. The ridges may extend at a non-zero angle to the longitudinal axis. The ridges may be non-perpendicular to the longitudinal axis. The second body may include a shank that extends distally from the grip to be movably received within the lumen of the first body. A tapered surface may extend between the grip and the shank.

In a further example, a medical device handle may comprise: a first body defining a lumen; and a second body having a portion that is movably received within the lumen. The second body may include a grip. The grip may include a plurality of ridges. The ridges may extend at a non-zero angle to a longitudinal axis of the second body. The ridges may be are non-perpendicular to the longitudinal axis. The second body may be configured to move proximally and distally along a longitudinal axis of the second body in order to actuate an element of a medical device.

Any of the devices disclosed herein may include any of the following features, alone or in any combination or subcombination. The longitudinal axis may be a central longitudinal axis. A first ridge of the plurality of ridges may have a first portion that extends a first radial distance from the central longitudinal axis. The first ridge may have a second portion that extends a second radial distance from the central longitudinal axis. The second radial distance may differ from the first radial distance. A proximal end of the second body may include an adapter for coupling to a fluid source. The adapter may include an opening defined by a first wall, a second wall surrounding the first wall, and at least one fin extending between the first wall and the second wall.

In another example, a medical device handle may comprise: a first body defining a lumen. The first body may include a first flange having a first concave surface. The first concave surface may face at least partially distally. The first body may include a second flange proximal of the first flange and having a second concave surface. The second concave surface may face at least partially proximally. The medical device handle may include a second body having a portion that is movably received within the lumen. The second body may be configured to move proximally and distally along a longitudinal axis of the second body in order to actuate an element of a medical device.

Any of the devices disclosed herein may include any of the following features, alone or in any combination or subcombination. The first portion may include a barrel having a plurality of ridges thereon. The barrel may be proximal of both the first flange and the second flange.

### BRIEF DESCRIPTION OF THE DRAWINGS

The accompanying drawings, which are incorporated in and constitute a part of this specification, illustrate aspects this disclosure and together with the description, serve to explain the principles of the disclosure.

FIG. 1A depicts an exemplary medical device in a first configuration.

FIG. 1B depicts a cross-section of a handle of the exemplary medical device of FIG. 1A in a second configuration.

FIGS. 2A-2B depict the exemplary medical device in the second configuration, being gripped by a hand of an operator.

FIGS. 3A-3B depict the exemplary medical device in the first configuration, being gripped by a hand of an operator.

FIGS. 4A-4B depict aspects of a proximal end of the medical device.

FIGS. 5A-5B show the exemplary medical device being gripped by one hand (FIG. 5A) or two hands (FIG. 5B) of an operator, with the medical device in a first orientation.

FIGS. 6A-6B show the exemplary medical device being gripped by one hand (FIG. 6A) or two hands (FIG. 6B) of an operator, with the medical device in a second orientation.

### DETAILED DESCRIPTION

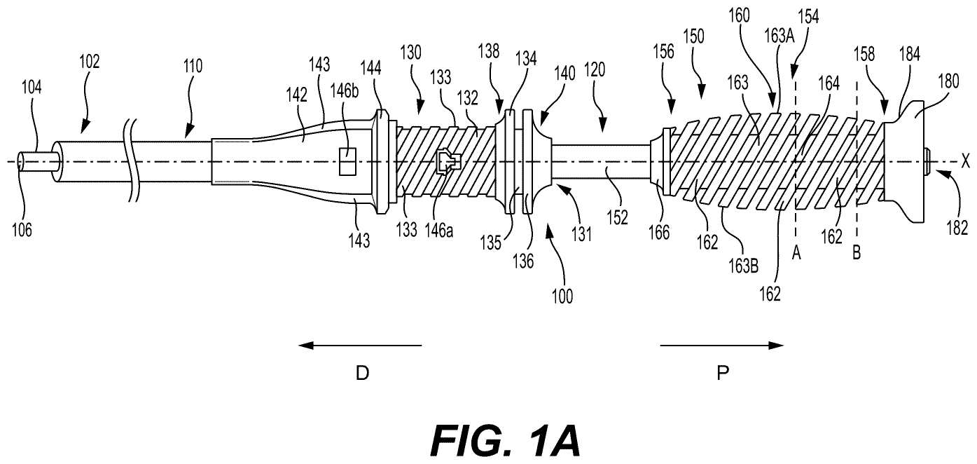

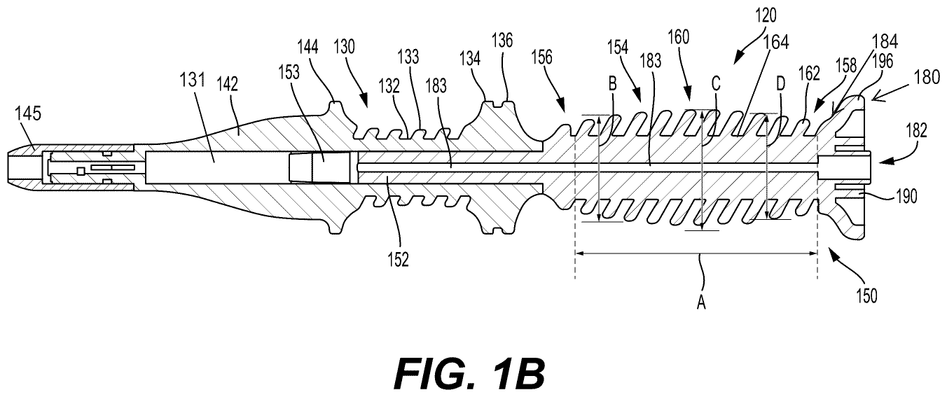

It may be understood that both the foregoing general description and the following detailed description are exemplary and explanatory only and are not restrictive of the invention, as claimed. As used herein, the terms “comprises,” “comprising,” “has,” “having,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator, and the term “proximal” refers to a direction toward an operator. FIG. 1A includes arrows labeled “P” and “D” to indicate proximal and distal directions, respectively. The term “exemplary” is used in the sense of “example,” rather than “ideal.” The term “approximately,” or like terms (e.g., “substantially”), includes values +/−10% of a stated value. Unless otherwise stated, ranges disclosed herein include the end points of the ranges.

Medical devices, such as injection needles, may include a handle for being gripped by an operator and a shaft distally extending from the handle. The shaft may be configured to be inserted into a body of a subject (e.g., via an introduction device such as an endoscope or other type of scope). A feature/element at the distal end of the shaft (e.g., a needle) may be operable to perform a procedure at a treatment site within the body of the subject. The handle may include an first body and a second body. The first body may define a lumen, in which a portion of the second body may be movably received. The second body may be extended and retracted relative to the first body in order to control the feature at the distal end of the shaft. The second body may include a grip for grasping by the operator in order to extend or retract the second body. The first body may include a finger rest for receiving fingers of the operator. The second body and the finger rest may be configured to be used by a variety of operators, and operators may hold the handle in various manners. The handle may be comfortable for an operator to hold and actuate, and the handle may include one or more features that facilitate an operator in locating a grip position, for example, without seeing the handle. Furthermore, the second body and/or the finger rest may include features to help prevent slip within the operator's hand. A proximal end of the second body may include an interface for receiving and securing a syringe or other source of fluid.

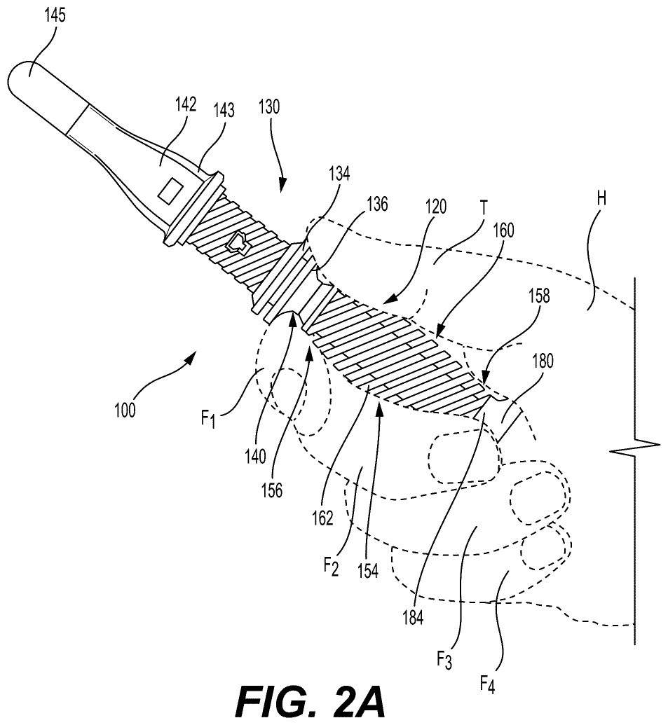

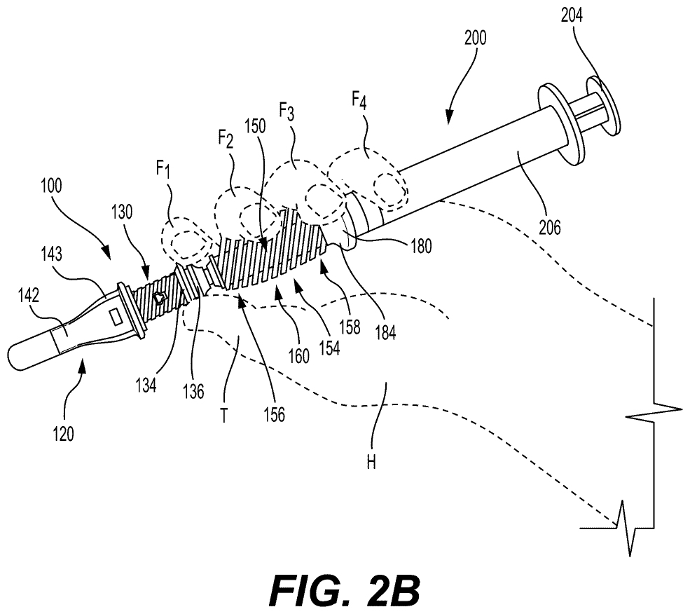

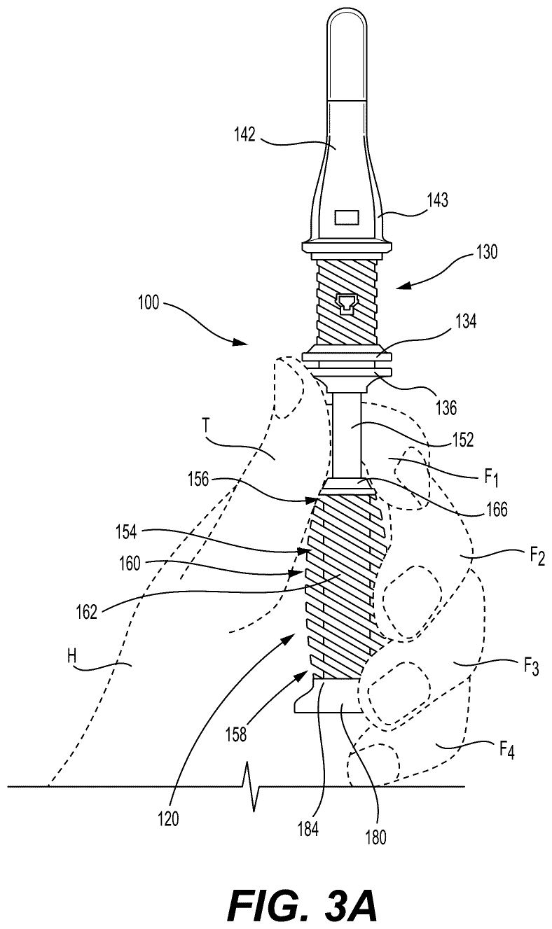

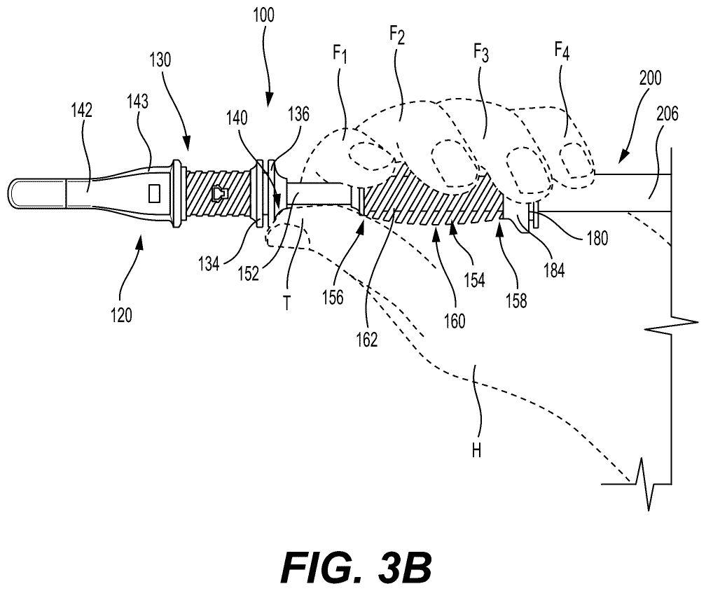

FIG. 1A shows a medical device 100 in a first configuration. FIG. 1B shows a handle 120 of medical device 100 in a second configuration. FIGS. 2A-3B show various views of medical device 100 in a hand H of an operator. With particular reference to FIG. 1A, medical device 100 may include a shaft 110 and a handle 120. Shaft 110 may have a distal end 102 with an element/feature configured to be operated by handle 120. For example, shaft 110 may have a needle 104 at its distal end 102. Needle 104 may be movable with respect to a sheath of shaft 110, such that needle 104 may be extended from (e.g., to a position distally beyond) a distal end of shaft 110 or retracted such that needle 104 is covered by shaft 110. Needle 104 or another element (e.g., a hypotube) may extend through shaft 110 to a proximal end of shaft 110. Needle 104 (or another element) may have a lumen 106 extending therethrough. As discussed below, a portion of handle 120 may be coupled to needle 104 or another element, and needle 104 or another element may be controlled with handle 120. Although a needle 104 is described herein, it will be appreciated that medical device 100 may include other types of features at its distal tip (e.g., basket, snare, forceps, stapler, etc.), and medical device 100 is not limited by a type of instrument at its distal tip.

Handle 120 may include a first body 130, which may be a distal portion of handle 120. Handle 120 may further include a second body 150, which may be a proximal portion of handle 120. First body 130 may define a lumen 131 for receiving a portion of second body 150 therein. For example, second body 150 may include a shank 152 that is movably received within lumen 131. First body 130 may be fixed with respect to shaft 110. Second body 150 may be akin to a plunger and may be movable proximally and distally relative to first body 130. As second body 150 moves proximally and distally, needle 104 may be retracted and advanced, respectively, for example, relative to distal end 102. For example, shank 152 or another element of second body 150 may be directly or indirectly coupled to needle 104. Although FIG. 1A is depicted with second body 150 moved relatively proximally relative to first body, needle 104 is shown as extending from a distal tip of shaft 110 in order to depict the elements of medical device 100. It will be appreciated that, in the configuration of handle 120 shown in FIG. 1A, needle 104 may in reality be fully retracted within shaft 110. FIG. 1B shows second body 150 in an advanced configuration, with second body 150 moved distally relative to first body 130.

First body 130 may include a barrel 132, which may serve as a finger rest for one or more fingers of an operator's hand. Barrel 132 may be approximately cylindrically shaped. A central longitudinal axis of barrel 132 may be approximately coaxial with a central longitudinal axis X of handle 120. Lumen 131 may extend longitudinally through barrel 132.

Barrel 132 may include a plurality of ridges 133 thereon. Ridges 133 may be raised with respect to other portions of barrel 132. As shown in FIG. 1B, ridges 133 may be formed integrally with other portions of barrel 132. Ridges 133 may extend spirally or diagonally along barrel 132. In other words, ridges 133 (along their apexes) may not extend perpendicularly to or parallel to central longitudinal axis X of handle 120 (may extend at a non-zero angle relative to central longitudinal axis X and may be non-perpendicular to central longitudinal axis X). In some examples, ridges 133 may extend at an angle between approximately 30 degrees and approximately 60 degrees relative to longitudinal axis X. The diagonal pattern of ridges 133 may help to reduce operator fatigue as compared to other shapes of ridges 133 (e.g., as compared to ridges that extend perpendicularly to a central longitudinal axis of a handle).

Radially outer surfaces of ridges 133 may have any suitable shape. For example, ridges 133 may have rounded outer surfaces or flat outer surfaces. Rounded outer surfaces may help to improve friction of a grip on barrel 132 and/or may help to decrease fatigue for an operator. As shown in FIG. 1B, in particular, along a line extending from a radially inner end of ridge 133 to a radially outer edge of ridge 133, ridges 133 may extend distally on one side of barrel 132 (e.g., a bottom side of barrel 132 in FIG. 1B) and proximally on another side of barrel 132 (e.g., a top side of barrel 132 in FIG. 1B). Ridges 133 may be approximately parallel to one another. Each of ridges 133 may have a same radial width (in a direction perpendicular to central longitudinal axis X) as the others of ridges 133. Each ridge 133 may have a uniform radial width along an entirety of ridge 133. Ridges 133 may help to reduce slip of first body 130 in an operator's hand, as compared to devices that lack such ridges. Because an operator may move second body 150 proximally and distally (thereby actuating needle 104) relative to first body 130 more than ten times in a single procedure, such anti-slip properties may help to reduce operator burden and fatigue.

First body 130 may further include a first flange 134 and a second flange 136. Each of flanges 134 and 136 may be proximal of ridges 133 on barrel 132. Flanges 132 and 134 may extend circumferentially around first body 130 (e.g., around an entire circumference of first body 130). A radius of flange 134, 136 may be uniform around an entire circumference of the respective flange. Central longitudinal axis X of handle 120 may extend through centers of first flange 134 and/or second flange 136. Although two flanges 134, 136 are shown, it will be appreciated that first body 130 may have any suitable number of flanges (e.g., one flange or more than two flanges). As shown in FIG. 1B, flanges 134 and 136 may be integrally formed with barrel 132. A proximal portion 135 of barrel 132 may extend between flanges 134 and 136. Alternatively, proximal portion 135 may be separate from barrel 132. First flange 134 may be distal to second flange 136.

First flange 134 may include a tapered portion 138 extending radially inward in a distal direction. Tapered portion 138 may at least partially face in a generally distal direction. Second flange 136 may include a tapered portion 140 extending radially inward in a proximal direction. Tapered portion 140 may at least partially face in a generally proximal direction. Tapered portions 138, 140, may each be curved. For example, as shown in FIG. 1A, tapered portions 138 and 140 may have a concave shape. Alternatively, one or more of tapered portions 138, 140 may have a convex shape or may taper linearly. As discussed in further detail below, flanges 134 and/or 136 may serve as finger rests for one or more fingers of an operator's hand. Additionally, flanges 134 and/or 136 may serve as positioning aids that assist an operator in positioning his or her hand without seeing handle 120.

First body 130 may also include a distal end 142. A central longitudinal axis of distal end 142 may be approximately coaxial with central longitudinal axis X of handle 120. Distal end 142 may taper radially inward in a distal direction, toward shaft 110. A proximal end portion of distal end 142 may include a third flange 144 (labeled in FIG. 1A) that extends radially outward. Distal end 142 may also include a plurality of ribs 143, extending in an approximately proximal/distal direction along distal end 142 (e.g., parallel to central longitudinal axis X). As shown in FIG. 1B, distal end 142 may be integrally formed with barrel 132 (and flanges 134, 136). Although FIGS. 1B-3B depict distal end 142 as terminating in a cap 145 (labeled in FIGS. 1B and 2A) at a distal end thereof, instead of shaft 110, it will be appreciated that cap 145 is shown merely for convenience of depicting features of handle 120 without shaft 110.

First body 130 may further include one or more locking mechanisms 146a, 146b (see FIG. 1A). For example, a first locking mechanism 146a may be positioned on barrel 132. A second locking mechanism 146b may be disposed on distal end 142. Locking mechanisms 146a, 146b may be utilized to selectively retain handle 120 in a position where needle 104 is advanced or retracted. For example, first locking mechanism 146a may interact with a feature of second body 150 (e.g., a portion of shank 152 that extends within first body 130) to help to retain second body 150 and needle 104 in retracted position. Second locking mechanism 146b may interact with a feature of second body 150 (e.g., a same or different portion of shank 152 that extends within first body 130) to help to retain second body 150 and needle 104 in an advanced position.

As mentioned, second body 150 may include shank 152. Shank 152 may have a rod/shaft shape. In some examples, shank 152 may be cylindrical in shape. A central longitudinal axis of shank 152 may be coaxial with central longitudinal axis X of handle 120. As discussed above, shank 152 may be received in and movable with respect to first body 130. In some examples, shank 152 or another element coupled to shank 152 may be coupled to needle 104 and/or a control element, such as a wire or cable, or any other suitable structure for controlling an element at distal end 102 of shaft 110. For example, as shown in FIG. 1B, shank 152 may include a coupler 153 for coupling to needle 104 and/or a control element.

Shank 152 may be fixedly coupled to a grip 154. In some examples, as shown in FIG. 1B, shank 152 may be integrally formed with grip 154. As discussed below, and shown with respect to FIGS. 2A-3B, an operator may hold grip 154 in order to move grip 154 and shank 152 relative to first body 130. Grip 154 may have a distal portion 156, a proximal portion 158, and a middle portion 160 between (e.g., at a midpoint between) distal portion 156 and proximal portion 158. In some examples, as shown in the Figures, grip 154 may bulge outward at middle portion 160, so that grip 154 has a convex overall shape along the proximal/distal directions. In other words, distal portion 156 and proximal portion 158 may have a smaller diameter/width relative to middle portion 160 (and middle portion 160 may have a greater diameter/width relative to distal portion 156 and proximal portion 158). In some examples, distal portion 156 may have approximately the same diameter/width as proximal portion 158. Alternatively, one of distal portion 156 or proximal portion 158 may have a smaller diameter than the other of distal portion 156 or proximal portion 158. For example, distal portion 156 may have a cross-sectional width/diameter B (perpendicular to longitudinal axis X of FIG. 1A) and proximal portion 156 may have a width/diameter D (perpendicular to longitudinal axis X of FIG. 1A). In some examples, width B and width D may be the same or approximately the same. In one example, widths B and D may be approximately 17.5 mm. Middle portion 106 may have a width/diameter C (perpendicular to longitudinal axis X of FIG. 1A) of approximately 19.7 mm. An axial length A (along or parallel to longitudinal axis X) of grip 154 may be approximately 40 mm. A shape of grip 154 (e.g., the bulging shape of grip 154) may help to provide increased ergonomic comfort to operators, as compared to other shapes.

Grip 154 may include a plurality of ridges 162 and a core 164. Ridges 162 may have any of the features of ridges 133, discussed above. Core 164 may have a cylindrical shape that may have an approximately uniform diameter/width. A central longitudinal axis of core 164 may be approximately coaxial with central longitudinal axis X of handle 120. Alternatively, core 164 may have a shape that varies along its length (e.g., bulges radially outward at middle portion 160). As shown in FIG. 1B, core 164 may be integrally formed with ridges 162 in some examples.

Ridges 162 may extend radially outward from core 164 and spirally or diagonally along core 164. In other words, ridges 162 may not be perpendicular to or parallel to central longitudinal axis X of handle 120. In some examples, ridges 162 may extend at an angle between approximately 30 degrees and approximately 60 degrees relative to central longitudinal axis X. Radially outer surfaces of ridges 162 may have any suitable shape. For example, ridges 162 may have rounded outer surfaces or flat outer surfaces. Rounded surfaces of ridges 162 may help to increase friction/grip between an operator and ridges 162 and/or may help to decrease fatigue for the operator. Ridges 162 may be approximately parallel to one another. Ridges 162 may also be approximately parallel to ridges 133 of barrel 132. Ridges 162 may help to reduce slip of grip 154 in an operator's hand, as compared to devices that lack such ridges. Because an operator may move second body 150 proximally and distally (thereby actuating needle 104) more than ten times in a single procedure, such anti-slip properties may help to reduce operator burden and fatigue. The diagonal pattern of ridges 162 may help to reduce operator fatigue as compared to other shapes of ridges 162 (e.g., as compared to ridges that extend perpendicularly to a central longitudinal axis of a handle).

In some examples, a cross-section of grip 154 that is perpendicular to central longitudinal axis X may be circular. It will be appreciated that, when describing a shape of grip 154 overall (e.g., the convex shape of grip 154 described above, or the cross-sectional shapes described herein), that the shape refers to an overall shape of grip 154, not accounting for the spaces between ridges 162. In other words, the shapes are described as if a surface extended along the radially outer surfaces of ridges 162. It will be appreciated that any actual cross-section of grip 154 would be affected by a location of ridges 162 in that cross-section. However, for ease of description, an overall shape of grip 154 is described. Alternatively, a cross-section of grip 154 perpendicular to central longitudinal axis X may have an oblong shape (e.g., an elliptical or ovular shape). In further alternatives, a cross-section of grip 154 perpendicular to central longitudinal axis X may have any suitable shape.

Ridges 162 may extend outward from core 164 by a varied amount, such that middle portion 160 of grip 154 has a larger diameter, as described above. Ridges 162 near distal portion 156 and/or proximal portion 158 may have a smaller radial dimension/thickness (a dimension perpendicular to central longitudinal axis X) than ridges near middle portion 160. A single ridge 162 may have a varying radial thickness (a radial distance that ridge 162 extends from core 164) along that ridge 162. For example, an exemplary ridge 163 (see FIG. 1A) of ridges 162 may have a first side 163A that is proximal to a second side 163B (opposite to first side 163A). First side 163A may be closer to middle portion 160 of grip 154 than second side 163B is. First side 163A may extend further radially outward by a greater radial distance (relative to central longitudinal axis X and core 164) than second side 163B does. In other words, a radial thickness of first side 163A may be greater than a radial thickness of second side 163B. Although first side 163A and second side 163B are labeled, it will be appreciated that a radial thickness of ridge 163 may vary throughout ridge 163. For grips 154 that have a circular cross-sectional shape, radial thickness of ridge 163 may taper outward from second side 163B to first side 163A. For grips 154 that have alternative cross-sectional shapes, it will be appreciated that a radial thickness of ridge 163 may be modulated in order to create the desired cross-sectional shape. It will be appreciated that ridge 163 is discussed merely for illustrative purposes and that some or all of ridges may have varying radial thicknesses, depending on their positions on grip 154.

For embodiments in which a cross-section of grip 154 has a round/circular shape, at a given axial location on grip 154, portions of ridges 162 at that position may have the same radial thickness. For example, line A on FIG. 1A denotes a first, exemplary, axial position along grip 154. Portions of multiple ridges 162 may extend through the first axial position. Those portions may all have the same radial thickness at a location of line A. Line B on FIG. 1A denotes a second, exemplary, axial position along grip 154. Similarly to the first axial position (line A), multiple ridges may be at the second axial position (line B). Portions of the ridges that extend through the second axial position may all have the same radial thickness at the second axial position (line B).

In alternatives, core 164 may bulge radially outward to create the curved shape of grip 154, and ridges 162 may have uniform radial thicknesses. In a further alternative, core 164 may bulge somewhat radially outward to partially create the curved shape of grip 154, and ridges 162 may have varying radial thicknesses to account for a remainder of the curved shape of grip 154. Core 164 and ridges 162 may have any suitable combined properties/shapes to create the curved profile of grip 154.

A tapered shoulder 166 may extend between shank 152 and grip 154, for example, from distal portion 156 of grip 154. Shoulder 166 may taper radially inward in a distal direction. Shoulder 166 may be curved in a concave shape. Alternatively, shoulder 166 may have an alternative shape, such as a convex shape or a linearly tapering shape. Shoulder 166 may at least partially transition between the smaller diameter of shank 152 to the larger diameter of grip 154.

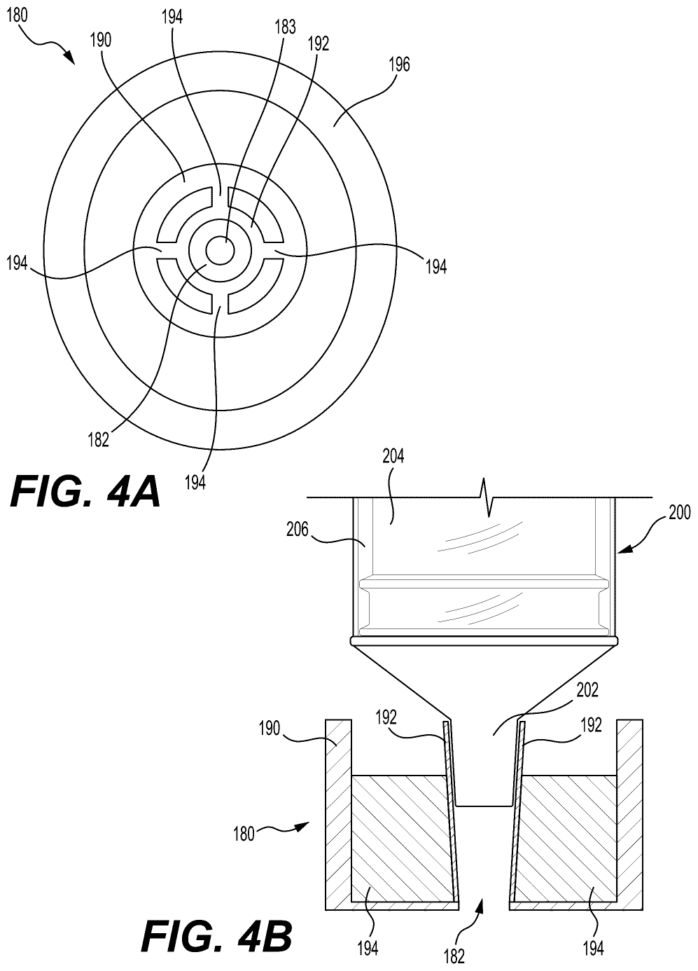

Second body 150 may further include an adapter 180 at a proximal end of second body 150, for example, proximal of proximal portion 158 of grip 154. Details of adapter 180 are further shown in FIGS. 4A and 4B. FIG. 4A shows a proximalmost end of adapter 180. FIG. 4B is a schematic longitudinal cross-sectional view of a portion of adapter 180. FIGS. 4A and 4B may not show all the details of shapes of adapter 180 but serve to illustrate features of adapter 180. Adapter 180 may be configured to couple a fluid source 200 to medical device 100. As shown particularly in FIGS. 1A and 1B, adapter 180 may have a tapered surface 184. Tapered surface 184 may taper in a proximal direction from a diameter/width that is the same as or similar to a diameter of proximal portion 158 of grip 154 to a larger diameter/width. Tapered surface 184 may have a concave shape. Alternatively, tapered surface 184 may have a convex shape or a linearly tapering shape, or any other suitable shape. As shown in FIG. 4A, an outer wall 196 of adapter 180 (which may include tapered surface 184) may have an oblong (e.g., approximately ovular or approximately elliptical) shape when viewing a proximal end of adapter 180 in plan view.

As shown in FIGS. 1A, 1B, 4A, and 4B, adapter 180 may have an opening 182, which may be configured to receive fluid source 200, as described below. Opening 182 may be in fluid communication with a lumen 106 of needle 104, such that fluid injected into opening 182 (e.g., via depressing a plunger 204 of fluid source 200 through a barrel 206 of fluid source 200) may be delivered to tissue via a distal opening of lumen 106. Opening 182 also may include a valve 183. Valve 183 may be a one-way valve that allows fluid to flow distally through lumen 106 from opening 182 but helps to prevent fluid from traveling proximally out of opening 182.

As shown in FIGS. 4A and 4B, opening 182 may be defined by an opening wall 192 (e.g., a first wall). Opening wall 192 may be tubular and may have an approximately circular shape when viewing a proximal end of adapter 180 in a plan view. A support wall 190 (e.g., a second wall) may extend around opening wall 192. Support wall 190 may also be tubular and may have an approximately circular shape when viewing a proximal end of adapter 180 in plan view. In some examples, opening wall 192 and support wall 190 may be concentric. Support wall 190 may have a larger diameter than opening wall 192. In some examples, as shown in FIGS. 4A and 4B, support wall 190 may have a greater thickness in a radial direction than opening wall 192 does. Outer wall 196 (not shown in FIG. 4B) may surround support wall 190. In some examples, outer wall 196 may be concentric with support wall 190 and opening wall 192. As shown in FIG. 4A, outer wall 196 may have a different shape than support wall 190 and/or opening wall 192 when viewing a proximal end of adapter 180 in a plan view. For example, outer wall 196 may be oblong, as described above, while support wall 190 and opening wall 192 may be circular or generally round. In some examples, as shown in FIG. 1B, outer wall 196 and support wall 190 may be formed integrally with grip 154 and plunger 152.

A plurality of ribs/fins 194 may extend between and be coupled to opening wall 192 and support wall 190. Fins 194 may extend radially outward relative to central longitudinal axis X (shown in FIG. 1A). Each fin 194 may be perpendicular to opening wall 192 and support wall 190 at the point where fin 194 joins opening wall 192/support wall 190. As shown in FIG. 4A, adapter 180 may include four fins 194. However, such a number is merely exemplary, and any suitable number of fins 194 may be utilized. In some aspects, fins 194 may be evenly spaced (e.g., spaced at approximately 90 degree angles for embodiments with four fins 194, spaced at approximately 120 degree angles for embodiments with three fins 194, etc.). In other aspects, fins 194 may be unevenly spaced around opening wall 192 and/or from support wall 190. In some examples, opening wall 192, support wall 190, and fins 194 may have approximately the same axial lengths (lengths parallel to central longitudinal axis X, in a proximal/distal direction). In some examples, fins 194 may be uniform to one another. In alternatives, fins 194 may have varying properties. In examples where fins 194 have varying properties, fins 194 may have variable spacing with one another (e.g., a thicker fin 194 may be spaced further apart than thinner fins 194).

In some examples, opening wall 192, support wall 190, and fins 194 may be monolithically formed from a single piece of material (e.g., molded, formed via additive manufacturing, machined, etc.). In alternatives, some or all of opening wall 192, support wall 190, and fins 194 may be formed from separate pieces of material that are coupled together (e.g., via adhesive, overmolding, or other mechanisms). In some examples, outer wall 196 may also be formed monolithically with opening wall 192, support wall 190, and fins 194.

As shown in FIG. 4B, a tip 202 of fluid source 200 (e.g., a syringe) may be inserted into opening 182. In FIG. 4B, a portion of adapter 180 is shown in cross-section, but fluid source 200 is not shown in cross-section. Fluid source 200 may be a straight syringe (e.g., not a Luer syringe), such that tip 202 lacks threads, ridges, etc. As shown in FIG. 4B, opening wall 192 may be sized so that opening 182 has a slightly smaller diameter than tip 202 of syringe. As tip 202 is inserted into opening 182, tip 202 may slightly deform opening wall 192. Fins 194 may exert a radially inward force on opening wall 192 and, thus, on tip 202, in order to help to retain tip 202 within opening 182. Adapter 180 may be relatively stronger than adapters that lack support wall 190 and fins 194, thereby helping to increase a hold/retention force on tip 202. Support wall 190 and fins 194 may have any suitable properties so as to exert a desired retaining force on tip 202 positioned within opening 182, while allowing tip 202 to be inserted into opening 182. For example, support wall 190 and/or opening wall 192 may extend further proximally than fins 194 to help insertion of tip 202 into opening 182.

FIGS. 2A-3B show a hand H of an operator holding medical device 100 in various positions. In the figures, T labels a thumb. F1, F2, F3, and F4 label first, second, third, and fourth fingers, respectively. The term “top knuckle” herein refers to a knuckle of a finger closest to the tip of the finger. The term “base knuckle” refers to a knuckle of a finger at the base of the finger. The term “middle knuckle” refers to the knuckle between the base knuckle and the top knuckle. For the thumb T, the term “base knuckle” refers to a knuckle at the base of the thumb, and the term “top knuckle” refers to the knuckle of the thumb that is between the base knuckle and the tip of the thumb. FIGS. 2A-2B show medical device 100 in a first, extended configuration (with needle 104 extended). FIGS. 3A-3B show medical device 100 in a second, retracted configuration (with needle 104 retracted). It will be appreciated that the positions of the hands shown in FIGS. 2A-3B are merely exemplary, and that operators may adopt a variety of one- or two-handed grips on medical device 100. Medical device 100 provides a comfortable, ergonomic grip to operators with varying hand shapes/sizes and grip preferences.

As shown in FIG. 2A, a portion of the operator's thumb T between the top knuckle and a tip of thumb T may rest on first flange 134 and/or second flange 136. An portion of operator's first finger F1 between the top knuckle and the middle knuckle may also rest on second flange 136. For example, that portion of the operator's first finger F1 (index finger) may rest on tapered portions 140 of second flange 136. The portion of the operator's first finger F1 that is between the tip of that finger and the top knuckle of that finger may rest on ridges 162 of distal portion 156 of grip 154. A portion of the operator's second finger F2 (middle finger) that is below the top knuckle may wrap around middle portion 160 of grip 154. A portion of the operator's second finger F2 between the tip of the finger and the top knuckle may rest on distal portion 156 of second body 150. Ridges 162 of grip 154 may help an operator to retain grip on handle 120. A tip of the operator's second finger F2 may rest on tapered surface 184 of adapter 180.

As shown in FIG. 2B, a portion of the operator's thumb T that is between the tip of the thumb T and the top knuckle of the operator's thumb T may rest on first flange 134 and/or second flange 136. A tip of the operator's first finger F1 may also rest on first flange 134 and/or second flange 136. The operator's second finger F2 may wrap around grip 154 near middle portion 160 of grip 154. The operator's third finger F3 may wrap around grip 154 near proximal portion 158. A tip of the third finger F3 may rest on tapered surface 184 of adapter 180. The operator's fourth finger F4 may wrap around a barrel 206 of fluid source 200. During operation, the operator may depress plunger 204 of fluid source 200 in order to deliver fluid into opening 182.

FIGS. 3A and 3B show handle 120 in a configuration in which second body 150 has been moved proximally relative to FIGS. 2A and 2B. The configurations of handle 120 in FIGS. 3A and 3B may correspond to a retracted position of needle 104 (FIG. 1A). The grips of FIGS. 3A and 3B may be the same or similar grips shown with (FIG. 3B) or without (FIG. 3A) a tip 202 of fluid source 200 inserted into opening 182 of adapter 180. A portion of the operator's thumb T between the top knuckle and the tip of thumb T may rest on second flange 136. A portion of the operator's first finger F1 below the top knuckle may wrap around shank 152. A portion of the operator's first finger F1 between the tip and the top knuckle may rest on shoulder 166 (e.g., on a concave surface of shoulder 166, described above). The operator's second finger F2 may wrap around grip 154, such as a portion of grip 154 near middle portion 160 of grip 154. A portion of the operator's third finger F3 that is below the top knuckle may wrap around grip 154, such as a portion of grip 154 extending between middle portion 160 and proximal portion 158. A portion of the operator's third finger F3 that is between the tip and the top knuckle may contact ridges 162 of proximal portion 158 of grip 154. A tip of the user's third finger F3 may rest on tapered surface 184 of adapter 180. As shown in FIG. 3B, when tip 202 is inserted into opening 182, the operator's fourth finger F4 may wrap around barrel 206 of fluid source 200 and/or may selectively depress plunger 204 (see FIGS. 2B and 4B). For example, the operator may use the opposite hand or fingers of the same hand H to depress plunger 204.

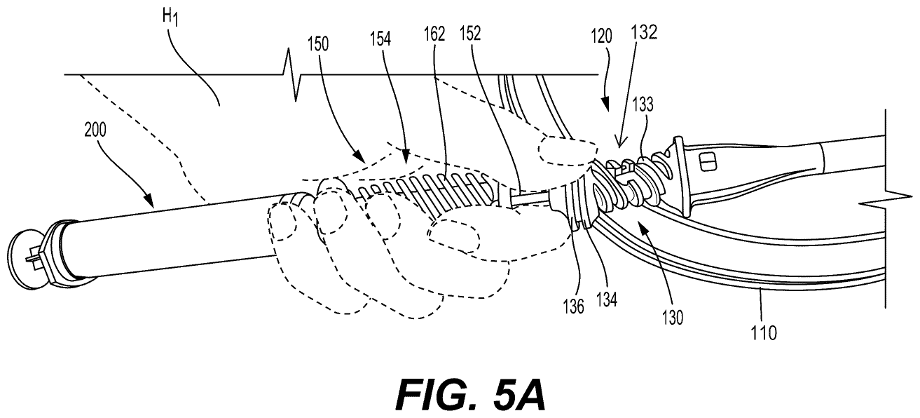

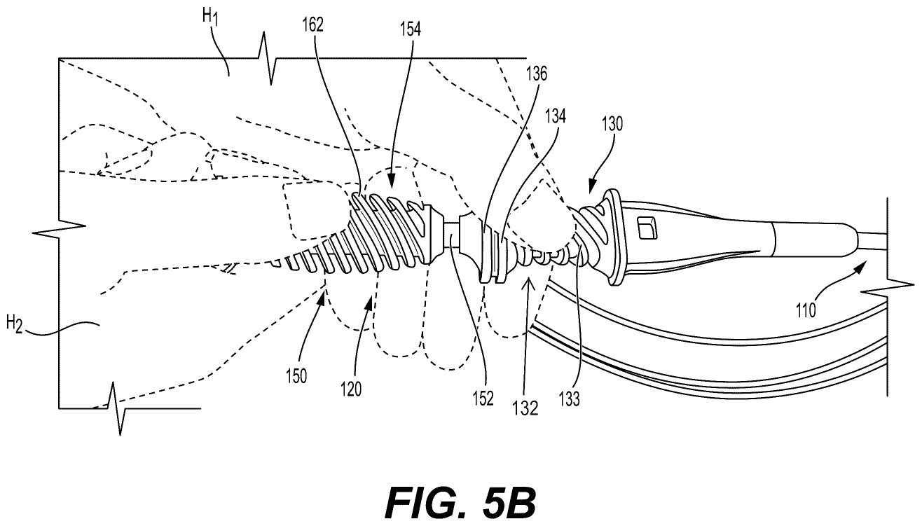

FIGS. 5A and 5B show exemplary manners of gripping medical device 100 when it is in a first orientation. In the first orientation, a top of medical device 100 (a side facing the operator and visible to the operator) may have ridges 162 and 133 extending diagonally across grip 154 and barrel 132, from one lateral side of device 100 to the other lateral side of device 100. As used herein, the term “top” refers to a position relative to an operator, instead of to a static portion of medical device 100. As shown in FIG. 5A, an operator may grip device 100 with one hand H1 (e.g., as described above). As shown in FIG. 5B, an operator may grip device 100 with two hands H1 and H2. The operator may utilize any position that is comfortable to the operator. As shown in FIGS. 5A and 5B, flanges 134 and 136 are easily accessible for resting one or more fingers thereon in the first orientation.

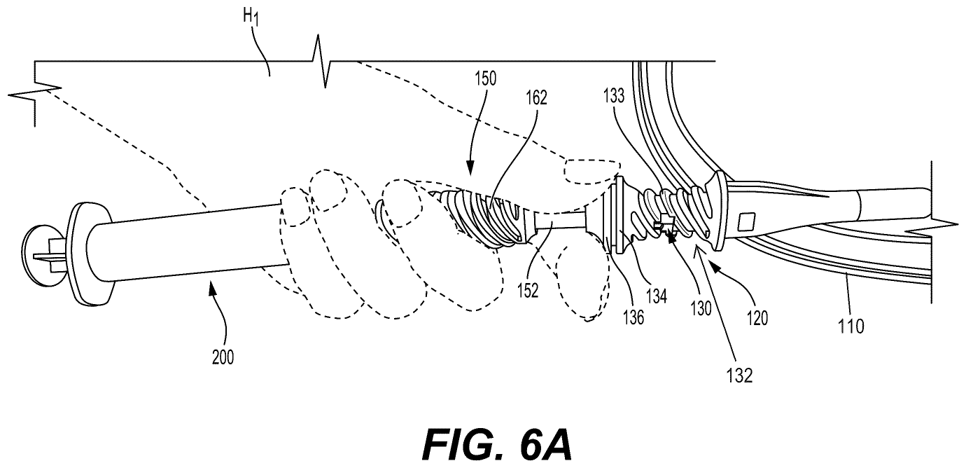

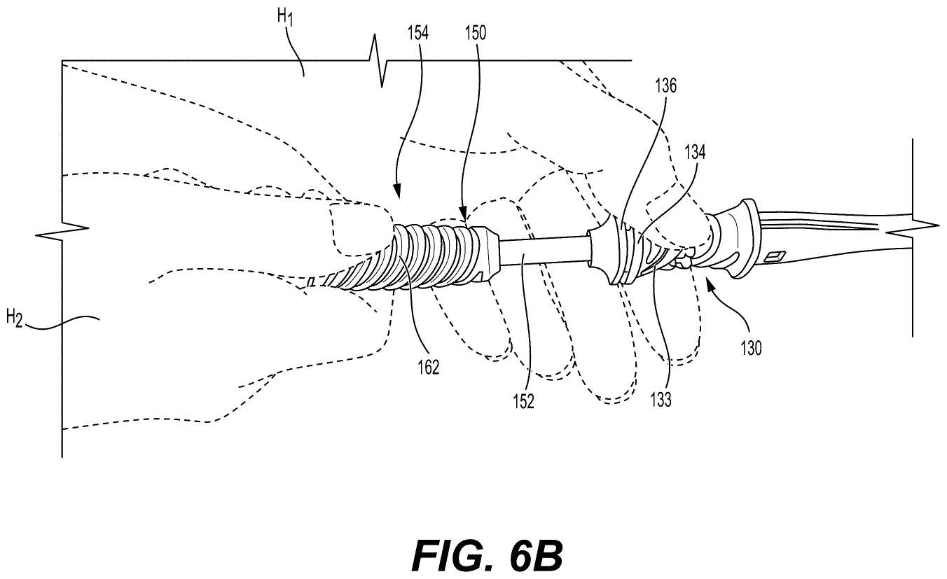

FIGS. 6A and 6B show exemplary manners of gripping medical device 100 when it is in a second orientation (FIG. 6A) and a third orientation (FIG. 6B). In the second and third orientations, device 100 may be rotated about axis X (FIG. 1A) relative to the first orientation. For example, the second orientation (FIG. 6A) may be rotated 90 degrees about axis X in a first direction relative to the first orientation (FIGS. 5A-5B). The third orientation (FIG. 6B) may be rotated 90 degrees about axis X in a second direction (opposite to the first direction) relative to the first orientation (FIGS. 5A-5B).

In the second and third orientations, a top of medical device 100 (a side facing the operator and visible to the operator) may have ridges 162 and 133 extending arcuately across grip 154 and barrel 132. As shown in FIG. 6A, in the second orientation, a center of the arcuate ridges 162, 133 may be proximal relative to sides of ridges 162, 133. As shown in FIG. 6B, in the third orientation, a center of the arcuate ridges 162, 133 may be distal relative to sides of ridges 162, 133. As shown in FIG. 6A, an operator may grip device 100 with one hand H1 (e.g., as described above). As shown in FIG. 6B, an operator may grip device 100 with two hands H1 and H2. The operator may utilize any position that is comfortable to the operator. As shown in FIGS. 6A and 6B, flanges 134 and 136 remain easily accessible for resting one or more fingers thereon in the second and third orientations. This differs from conventional devices, in which any flange may only be accessible to the user in certain orientations of the device. Although exemplary first, second, and third orientations are discussed above, it will be appreciated that flanges 134 and 136 are accessible to the operator for positioning and/or bracing in any orientation of device 100.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Additionally, a variety of elements from each of these embodiments can be combined to achieve a same or similar result as one or more of the disclosed embodiments. Accordingly, the invention is not to be considered as limited by the foregoing description.

We claim:

1. A medical device handle comprising:
a first body defining a lumen; and
a second body having a portion that is movably received within the lumen, wherein the second body includes a grip, wherein the grip includes a proximal portion, a distal portion, and a middle portion between the proximal portion and the distal portion, and wherein the middle portion has a greater cross-sectional width than the proximal portion and the distal portion;
wherein the second body is configured to move proximally and distally along a longitudinal axis of the second body in order to actuate a needle of a medical device.

2. The medical device handle of claim 1, wherein the grip includes a plurality of ridges.

3. The medical device handle of claim 2, wherein the ridges extend at a non-zero angle to the longitudinal axis, and wherein the ridges are non-perpendicular to the longitudinal axis.

4. The medical device handle of claim 2, wherein the longitudinal axis is a central longitudinal axis, and wherein a first ridge of the plurality of ridges has a first portion that extends a first radial distance from the central longitudinal axis, and wherein the first ridge has a second portion that extends a second radial distance from the central longitudinal axis, wherein the second radial distance differs from the first radial distance.

5. The medical device handle of claim 1, wherein the first body includes a flange at a proximal end thereof.

6. The medical device handle of claim 5, wherein the flange is a first flange, and wherein the first body includes a second flange that is proximal of the first flange.

7. The medical device handle of claim 6, wherein the first flange has a curved surface that faces distally and the second flange has a curved surface that faces proximally.

8. The medical device handle of claim 1, wherein a proximal end of the second body includes an adapter for coupling to a fluid source.

9. The medical device handle of claim 7, wherein the adapter includes an opening defined by a first wall, a second wall surrounding the first wall, and at least one fin extending between the first wall and the second wall.

10. The medical device handle of claim 9, wherein the at least one fin includes four fins that are evenly spaced from one another.

11. The medical device handle of claim 9, wherein the at least one fin extends radially outward from the first wall.

12. The medical device handle of claim 8, wherein the adapter includes a concave proximal surface.

13. The medical device handle of claim 1, wherein the first body includes a barrel having a plurality of ridges thereon.

14. The medical device handle of claim 13, wherein the ridges extend at a non-zero angle to the longitudinal axis, and wherein the ridges are non-perpendicular to the longitudinal axis.

15. The medical device handle of claim 1, wherein the second body includes a shank that extends distally from the grip to be movably received within the lumen of the first body, and wherein a tapered surface extends between the grip and the shank.

16. A medical device handle comprising:
a first body defining a lumen; and
a second body having a portion that is movably received within the lumen, wherein the second body includes a grip, wherein the grip includes a plurality of ridges, wherein the ridges extend at a non-zero angle to a longitudinal axis of the second body, and wherein the ridges are non-perpendicular to the longitudinal axis;
wherein the second body is configured to move proximally and distally along a longitudinal axis of the second body in order to actuate an element of a medical device.

17. The medical device handle of claim 16, wherein the longitudinal axis is a central longitudinal axis, and wherein a first ridge of the plurality of ridges has a first portion that extends a first radial distance from the central longitudinal axis, and wherein the first ridge has a second portion that extends a second radial distance from the central longitudinal axis, wherein the second radial distance differs from the first radial distance.

18. The medical device handle of claim 16, wherein a proximal end of the second body includes an adapter for coupling to a fluid source, and wherein the adapter includes an opening defined by a first wall, a second wall surrounding the first wall, and at least one fin extending between the first wall and the second wall.

19. A medical device handle comprising:
a first body defining a lumen, wherein the first body includes:
a first flange having a first concave surface, wherein the first concave surface faces at least partially distally; and
a second flange proximal of the first flange and having a second concave surface, wherein the second concave surface faces at least partially proximally; and
a second body having a portion that is movably received within the lumen;
wherein the second body is configured to move proximally and distally along a longitudinal axis of the second body in order to actuate an element of a medical device.

20. The medical device handle of claim 19, wherein the first portion includes a barrel having a plurality of ridges thereon, and wherein the barrel is proximal of both the first flange and the second flange.
